(19) United States
(12) Patent Application Publication
(10) Pub. No.: US 2005/0063995 A1
(43) Pub. Date: Mar. 24, 2005

(54) Compositions and methods for selected tumour treatment

(76) Inventors: David E Spaner, Toronto (CA); Arkady Mandel, North York (CA)

Correspondence Address: FOLEY & LARDNER LLP;THREE PALO ALTO SQUARE, 3000 EL CAMINO REAL, SUITE 100, PALO ALTO, CA, 94306, US

(21) Appl. No.: 10/493,544

(22) PCT Filed: Oct. 25, 2001
(86) PCT No.: PCT/CA02/01614

Related U.S. Application Data
(60) Provisional application No. 60/335,142, filed on Oct. 25, 2001.

(51) Int. Cl. A61K 39/00; C12N 5/08

(52) U.S. Cl. 424/277.1; 435/366

(57) ABSTRACT

Disclosed are novel compositions, methods and vaccines, which upon administration to a patient suffering from a melanoma, colon carcinoma tumor or breast cancer, postpone and/or reduce the need for chemotherapy treatment, slow the progression of or eliminate the tumor and/or alleviate the symptoms of the tumor. The compositions comprise stressed colon carcinoma, melanoma or breast cancer cells, preferably autologous such cells.

### FIELD OF THE INVENTION

This invention relates to the treatment of tumors, in particular to compositions and vaccines for the use in treating solid tumors, for example, colon carcinomas, melanomas and breast cancers.

### BACKGROUND OF THE INVENTION

All patents, patent applications, articles and publications mentioned herein, are hereby incorporated herein by reference in their entirety.

In spite of numerous advances in medical research, cancer remains a leading cause of death throughout the world. Non-specific approaches to cancer management, such as surgery, radiotherapy and generalized chemotherapy, have been successful in the management of a selective group of circulating and slow-growing solid cancers. However, many solid tumors are considerably resistant to such approaches, and the prognosis in such cases is correspondingly grave.

An emerging area of cancer treatment is immunotherapy. The general principle is to confer upon the subject being treated an ability to mount what is in effect a rejection response, specifically against the malignant cells. There are a number of immunological strategies under development, including: adoptive immunotherapy using stimulated autologous cells of various kinds; systemic transfer of allogeneic lymphocytes; intra-tumor implantation of immunologically reactive cells; and vaccination at a distant site to generate a systemic tumor-specific immune response.

The first strategy noted above, adoptive immunotherapy, is directed toward providing the patient with a level of enhanced immunity by stimulating cells ex vivo, and then readministering them to the patient. One previously used approach is to stimulate autologous lymphocytes ex vivo with tumor-associated antigen to increase numbers of tumor-reactive T-cells. The cells are histocompatible with the subject, and are generally obtained from a previous autologous donation.

The second strategy for cancer immunotherapy noted above is adoptive transfer of allogeneic lymphocytes. The rationale of this experimental strategy is to create a general level of immune stimulation, and thereby overcome the anergy that prevents the host's immune system from rejecting the tumor.

The third strategy is intra-tumor implantation. This is a strategy directed at delivering effector cells directly to the site of the tumor. Since the transplanted cells do not circulate, they need not be histocompatible with the host. Intra-tumor implantation of allogeneic cells may promote the ability of the transplanted cells to react with the tumor, and initiate a potent graft versus tumor response.

The fourth immunotherapy strategy is the generation of an active systemic tumor-specific immune response of host origin. The response is elicited from the subject's own immune system by administering a vaccine composition at a site distant from the tumor. The specific antibodies or immune cells elicited in the host as a result will hopefully migrate to the tumor, and then eradicate the cancer cells, wherever they are in the body. Various types of vaccines have been proposed, including isolated tumor-antigen vaccines and anti-idiotype vaccines. These approaches are based on the premise that tumors of related tissue type share a common tumor-associated antigen.

An alternative approach to an anti-tumor vaccine is to use tumor cells from the subject to be treated, or a derivative of such cells. In U.S. Pat. No. 5,484,596, Hanna Jr. et al. Claim a method for treating a resectable carcinoma to prevent recurrence or metastases, comprising surgically removing the tumor, dispersing the cells with collagenase, irradiating the cells using x-rays, and vaccinating the patient with at least three consecutive doses of about 10⁷ cells. The cells may optionally be cryopreserved, and the immune system may be monitored by skin testing. This approach does not solve the well-established observations that many tumors are not naturally immunogenic. Many patients from which tumors have been resected are either tolerant or unable to respond to their own tumor antigen, even when comprised in a vaccine preparation.

A suitable strategy for a human anti-tumor cellular vaccine has to contend with the following problems: a) heterogeneity amongst tumors (even tumors of the same type) in the display of tumor-associated antigens; b) heterogeneity in the immune response between individuals with regard to both antigens and cytokines; c) ethical and regulatory concerns about compositions that may be used in humans; and d) lack of development time in most clinical settings, limiting the ability to engineer new cell lines or otherwise tailor the vaccine to each patient.

Accordingly, a need exists for improved treatments of tumors which reduces the problems noted above and which also reduces the problems encountered with repeated use of chemotherapeutic agents having undesirable toxic side effects.

### SUMMARY OF THE INVENTION

This invention provides novel compositions, methods and vaccines, which upon administration to a patient suffering from a tumor selected from colon carcinoma, melanoma and breast cancer, postpone and/or reduce the need for chemotherapy treatment, slow the progression of or eliminate the tumor and/or alleviate one or more of the symptoms of the tumor. In some instances, they render the tumor more susceptible to treatment with conventional anti-cancer therapies (radiation, chemotherapy, etc.).

Accordingly, this invention provides compositions and methods for eliciting an anti-tumor response in a human patient in need thereof.

One embodiment of this invention is a pharmaceutical composition for administration to a mammalian patient suffering from a tumor selected from colon carcinoma, melanoma and breast cancer, said composition comprising autologous mammalian colon carcinoma and/or melanoma and/or beast cancer tumor cells wherein the autologous tumor cells have been treated so as to give rise to modification such that said autologous tumor cells are effective to elicit an immune response to the colon carcinoma and/or melanoma and/or breast cancer tumor in the mammal.

Another embodiment is a method for treating a patient having a tumor selected from colon carcinoma, melanoma and breast cancer, comprising administering to the patient the pharmaceutical composition defined above.

Yet another embodiment is a kit of parts comprising:
(a) tumor cells treated ex vivo with oxidative stress and UV light;
(b) a pharmaceutically acceptable excipient; and
(c) means for administering (b) and (c) to a tumor-suffering patient.

### REFERENCE TO THE DRAWINGS

FIGS. 1 and 2 of the accompanying drawings are graphical presentations of the results obtained according to Example 1 below;

FIGS. 3 and 4 are similar graphical presentations of the results obtained according to Example 2 below;

FIGS. 5, 6 and 7 are similar graphical presentations of the results obtained according to Example 3 below.

### DESCRIPTION OF THE INVENTION

It has been discovered that colon carcinoma and/or melanoma and/or breast cancer tumor cells stressed ex vivo with an oxidative stressor and UV light that are then administered to a mammalian patient suffering from a colon carcinoma, melanoma or breast tumor respectively, alleviate one or more of the symptoms of the respective tumor. The procedure, in one preferred embodiment, involves extracting an appropriate quantity of tumor cells from the patient, subjecting the tumor cells to an oxidative stressor and UV light, and reintroducing the same to the patient. In other embodiments, the source of the tumor cells is compatible mammalian donors or cultured cell lines, subjected to the same stressors.

The result, after one or more treatments, is a significant alleviation in one or more of the symptoms of the patient's tumor disease, as indicated by a reduced tumor size and/or load, a reduction in other tumor disease-related symptoms such as a reduction or elimination of metastasis, an increase in T-cells that secrete IFN-gamma after stimulation by tumor antigens, and/or an increased susceptibility of the tumor to treatment with conventional anti-cancer therapies such as radiation, chemotherapy, etc.

Prior to the further description of this invention, the following terms are defined:

The terms “vaccine,” “immunogen,” or “immunogenic composition” are used herein to refer to a compound or composition, as appropriate, that is capable of conferring a degree of specific immunity when administered to a human or animal subject. As used in this disclosure, a “cellular vaccine” or “cellular immunogen” refers to a composition comprising at least one cell population, which is optionally inactivated, as an active ingredient. The vaccines, immunogens, and immunogenic compositions of this invention are active vaccines, which means that they are capable of stimulating a specific immunological response (such as an anti-tumor antigen or anti-cancer cell response) mediated at least in part by the immune system of the host. The immunological response may comprise antibodies, immunoreactive cells (such as helper/inducer or cytotoxic cells), or any combination thereof, and is preferably directed towards an antigen that is present on a tumor towards which the treatment is directed. The response may be elicited or re-stimulated in a subject by administration of either single or multiple doses of vaccine.

A compound or composition is “immunogenic” if it is capable of either: a) eliciting an immune response; or b) reconstituting, boosting, or maintaining an immune response in an individual beyond what would occur if the compound or composition was not administered. A composition is immunogenic if it is capable of attaining either of these criteria when administered in single or multiple doses.

“Eliciting” an immune or immunological response, as the term is used herein, refers to administration of a compound or composition that initiates, boosts, or maintains the capacity for the host's immune system to react to a target substance, such as a foreign molecule, an allogeneic cell, or a tumor cell, at a level higher than would otherwise occur. Eliciting a “primary” immune response refers herein to eliciting specific immune reactivity in a subject in which previous reactivity was not detected; for example, due to lack of exposure to the target antigen, refractoriness to the target, or immune suppression. Eliciting a “secondary” response refers to the reinitiation, boosting, or maintenance of reactivity in a subject in which previous reactivity was detected; for example, due to natural immunity, spontaneous immunization, or treatment using one or several compositions or procedures.

A “cell line” or “cell culture” denotes higher eukaryotic cells grown or maintained in vitro. It is understood that the descendants of a cell may not be completely identical (either morphologically, genotypically, or phenotypically) to the parent cell.

“Inactivation” of a cell is used herein to indicate that the cell has been rendered incapable of cell division to form progeny. The cell may nonetheless be capable of response to stimulus, or biosynthesis and/or secretion of cell products such as cytokines. Methods of inactivation are known in the art. Preferred methods of inactivation are treatment with toxins such as mitomycin C, or irradiation using x-rays. Cells that have been fixed or permeabilized and are incapable of division are also examples of inactivated cells.

The terms “tumor cell” or “cancer cell,” used either in the singular or plural form, refer to cells of colon tumors and/or skin tumors and/or breast tumors that have undergone a malignant transformation that makes them pathological to the host organism. Preferably, the cells have up-regulated heat-shock proteins and/or tumor associated antigen. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells.

Tumors that can be treated by the compositions and methods of this invention are of three types: colon tumors, such as epithelial adenocarcinoma and their metastases; skin tumors, such as malignant melanoma, and breast tumors.

The term “tumor-associated antigen” or “TAA” is used herein to refer to a molecule or complex which is expressed at a higher frequency or density by tumor cells than by non-tumor cells of the same tissue type. Tumor-associated antigens may be antigens not normally expressed by the host; they may be mutated, truncated, misfolded, or otherwise abnormal manifestations of molecules normally expressed by the host; they may be identical to molecules normally expressed but expressed at abnormally high levels; or they may be expressed in a context or milieu that is abnormal. Tumor-associated antigens may be, for example, proteins or protein fragments, complex carbohydrates, gangliosides, haptens, nucleic acids, or any combination of these or other biological molecules. Knowledge of the existence or characteristics of a particular tumor-associated antigen is not necessary for the practice of the invention.

As used herein, “treatment” refers to clinical intervention in an attempt to alter the natural course of the individual or cell being treated, and may be performed either for prophylaxis or during the course of clinical pathology. Desirable effects include preventing occurrence or recurrence of disease, alleviation of symptoms, diminishment of any direct or indirect pathological consequences of the disease, preventing metastasis, lowering the rate of disease progression, amelioration or palliation of the disease state, and remission or improved prognosis.

The “pathology” associated with a disease condition is anything that compromises the well-being, normal physiology, or quality of life of the affected individual. This may involve, but is not limited to, destructive invasion of affected tissues into previously unaffected areas, growth at the expense of normal tissue function, irregular or suppressed biological activity, aggravation or suppression of an inflammatory or immunological response, increased susceptibility to other pathogenic organisms or agents, and undesirable clinical symptoms such as pain, fever, nausea, fatigue, mood alterations, and such other features as may be determined by an attending physician.

An “effective amount” is an amount sufficient to effect a beneficial or desired clinical result, particularly the generation of an immune response, or noticeable improvement in clinical condition. An immunogenic amount is an amount sufficient in the subject group being treated (either diseased or not) to elicit an immunological response, which may comprise either a humoral response, a cellular response, or both. In terms of clinical response for subjects bearing a neoplastic disease, an effective amount is amount sufficient to palliate, ameliorate, stabilize, reverse or slow progression of the disease, or otherwise reduce pathological consequences of the disease. An effective amount may be given in single or divided doses. Preferred quantities and cell ratios for use in an effective amount are given elsewhere in this disclosure.

An “individual,” “patient” or “subject” is a vertebrate, preferably a mammal, more preferably a human. Non-human mammals include farm animals and pets.

Compositions

The tumor cells of the compositions of this invention are colon carcinoma, melanoma and breast tumor cells which have been treated so as to give rise to modification such that the cells are effective to elicit an immune response to the melanoma, colon carcinoma or breast tumor, respectively, in the mammal. Once the tumor cells are administered to the patient, an immune response is elicited. The tumor cells used in the compositions and vaccines of this invention are treated ex vivo as described below in Methodology.

The compositions of the invention are tumor cell mixtures in physiologically compatible excipient, and are referred to herein as a vaccine or an immunogenic composition. They may be administered to patients either to treat or palliate a clinically detectable tumor, or for prophylaxis against resurgence of the tumor, particularly after surgical debulking, chemotherapy or radiation therapy of a previously detectable tumor. The compositions are typically administered at a location distant from the original tumor, with the objective of eliciting or stimulating a systemic reactivity against the primary tumor and metastases. The reactivity may, in turn, eradicate or slow the development of tumor cells, either at the primary site, within metastases (if there are any), or both.

It is preferred that the colon carcinoma, melanoma or breast tumor cells used in this invention are autologous tumor cells. However, it is contemplated that the tumor cells may also be allogeneic tumor cells. Mixtures of autologous and allogeneic tumor cells may also be used.

The autologous tumor cells are obtained by removal from the patient to be treated. The process for removing the cells from the patient is discussed below. In the case of allogeneic tumor cells, these may be obtained from suitable cultured cell lines. It may be desirable to further culture the autologous and/or allogeneic tumor cells prior to subjecting the cells to the various stressors described below in order to obtain a suitable number of cells needed to elicit an immune response in the patient.

The compositions of this invention may be used for administration to both human and non-human vertebrates. They provide advantages over previously available compositions particularly in spontaneous tumors. Veterinary applications are contemplated within the scope of the invention. The vaccines may be given to any human subject, with the discretion of the managing clinician, who will either have colon cancer, melanoma or breast cancer, or be at substantial risk of developing colon cancer, melanoma or breast cancer.

The vaccines prepared from the tumor cells may be stored prior to administration to a patient. They may be stored in frozen form, under refrigeration typically at about 4□C, frozen e.g. at −20° C. or in lyophilized form. Some stored vaccine may be retained for future use as “booster” shots after initial administration of the vaccine to the patient.

Typical human subjects for therapy comprise two groups, which may be distinguished by clinical criteria. Patients with “advanced disease” or “high tumor burden” are those who bear a clinically measurable tumor of the aforementioned type. A clinically measurable tumor is one that can be detected on the basis of tumor mass (e.g., by palpation, MRI, CAT scan, X-ray, or radioscintigraphy; positive biochemical or histopathological markers on their own are insufficient to identify this population).

A vaccine composition embodied in this invention is administered to patients with advanced disease with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of cancer, but have been responsive to another mode of therapy. The prior therapy may have included (but is not restricted to) surgical resection, radiotherapy, traditional chemotherapy, and other modes of immunotherapy. As a result, these individuals have no clinically measurable tumor of the aforementioned type by the definition given above. However, they are suspected of being at risk for recurrence or progression of the disease, either near the original tumor site, or by metastases. The adjuvant group may be further subdivided into high-risk and low-risk individuals. The subdivision is made on the basis of features observed before or after the initial treatment. These features are known in the clinical arts, and are suitably defined for each different cancer. Features typical of high risk subgroups are those in which the tumor has invaded neighboring tissues, or which show involvement of lymph nodes.

A vaccine composition embodied in this invention is administered to patients in the adjuvant group in order to elicit an anti-cancer response primarily as a prophylactic measure against recurrence. Ideally, the composition delays recurrence of the melanoma, colon cancer or breast cancer, or more preferably, reduces the risk of recurrence (i.e., improves the cure rate). Such parameters may be determined in comparison with other patient populations and other modes of therapy.

Of course, crossovers between these two patient groups occur, and the vaccine compositions of this invention may be administered at any time that is appropriate. For example, therapy may be conducted before or during traditional therapy of a patient with high tumor burden, and continued after the tumor becomes clinically undetectable. Therapy may be continued in a patient who initially fell in the adjuvant group, but is showing signs of recurrence.

The immune status of the individual may be any of the following: The individual may be immunologically naive with respect to certain tumor-associated antigens present in the composition, in which case the compositions may be given to initiate or promote the maturation of an anti-tumor response. The individual may not currently be expressing anti-tumor immunity, but may have immunological memory, particularly T cell memory relating to a tumor-associated antigen comprised in the vaccine, in which case the compositions may be given to stimulate a memory response. The individual may also have active immunity (either humoral or cellular immunity, or both) to a tumor-associated antigen comprised in the vaccine, in which case the compositions may be given to maintain, boost, or maturate the response, or recruit other arms of the immune system.

It is recognized that cancer patients often show a degree of immunosuppression, and this does not necessarily prevent the use of the compositions of the invention, as long as the compositions may be given safely and effectively. Immunocompetence in the subject may be of host origin.

Methodology

General Techniques

The practice of the present invention will employ, unless otherwise indicated, conventional techniques of molecular biology, microbiology, cell biology, biochemistry and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); “Current Protocols in Immunology” (J. E. Coligan et al., eds., 1991).

General procedures for the preparation and administration of pharmaceutical compositions are outlined in Remington's Pharmaceutical Sciences 18th Edition (1990), E. W. Martin ed., Mack Publishing Co., PA.

There are a number of well established animal models for tumor and tumor treatment that can be used to test and adjust the compositions and methods of this invention. Certain models involve injecting immune competent in-bred animals with established syngeneic tumor lines. The tumors can be co-injected with a potentially therapeutic composition, allowed to establish before therapy is commenced, or administered as a challenge at some time following vaccination of a naive animal.

Preparation of Cellular Vaccines

The cellular vaccines of this invention are typically assembled by preparing the cell population or equivalent thereof in an appropriate fashion, combining the components, and optionally storing cell mixtures before administration to a subject.

The compositions and methods of this invention provide for the prophylactic or therapeutic treatment of a tumor. The patient is evaluated to determine whether the tumor disease condition (melanoma, colon carcinoma or breast cancer) or risk of disease condition can be effectively treated by reducing or eliminating the tumor through the use of the treatment described herein. If a reduction or elimination of the tumor would be suitable for the prophylactic or therapeutic treatment of such tumor or tumor disease condition using the methods and compositions of the present invention, then the patient is administered tumor cells which have been treated ex vivo with an oxidative environment stressor and UV light, optionally also with a thermal stressor. The ex vivo treatment of the tumor cells is described below.

The tumor cells are administered to the mammal by a method suitable for vaccination selected from the group consisting of intra-arterial injection, intramuscular injection, intravenous injection, subcutaneous injection, intraperitoneal injection, and oral, nasal or rectal administration.

Ex Vivo Treatment of Tumor Cells

According to a preferred process of the present invention, tumor cells are extracted from a mammalian subject, preferably a human, having a tumor. The tumor cells are treated ex vivo with certain stressors, described in more detail below. The effect of the ex vivo stressors is to modify the tumor cell. The modified tumor cells are then re-introduced into the patient's body by any route suitable for vaccination.

The stressors to which the tumor cells are subjected ex vivo according to the method of the present invention are selected from temperature stress (blood temperature above or below body temperature), an oxidative environment and an electromagnetic emission, such as UV light, individually or in any combination, simultaneously or sequentially. Suitably, in human subjects, a sufficient number of treated tumor cells are administered such that, when re-introduced into the subject's body, at least partial alleviation of the symptoms of the tumor or a reduction in tumor size are achieved in the subject.

Tumor cells are removed from the patient by biopsy or other surgical procedures. A sufficient number of cells are removed for exposure to the various stressors noted above. Once cells are removed from the patient, they may be suspended in a biocompatible suspension media.

Preferably, the cells are suspended in a volume of up to about 400 ml, preferably from about 0.1 to about 100 ml, more preferably from about 5 to about 15 ml, even more preferably from about 8 to about 12 ml, and most preferably about 10 ml. A pharmaceutically acceptable excipient may be added.

It is preferred, according to the invention, to apply all three of the aforementioned stressors simultaneously to the tumor cells under treatment, in order to ensure the appropriate modification to the tumor cells. It may also be preferred in some embodiments of the invention to apply any two of the above stressors, for example to apply temperature stress and oxidative stress, temperature stress and an electromagnetic emission, or an electromagnetic emission and oxidative stress. Care must be taken to utilize an appropriate level of the stressors to thereby effectively modify the tumor cells to stimulate an immune response in the subject.

The temperature stressor warms the tumor cells being treated to a temperature above normal body temperature or cools the tumor cells below normal body temperature. The temperature is selected so that the temperature stressor does not cause excessive lysis in the tumor cells and so that, when the treated tumor cells are injected into a subject, alleviation of the tumor-related disease will be achieved. Preferably, the temperature stressor is applied so that the temperature of all or a part of the tumor cells is up to about 55° C., and more preferably in the range of from about −5° C. to about 36.5° C. (below body temperature), and 40° C. to about 55° C. (above body temperature).

In some preferred embodiments of the invention, the temperature of the tumor cells is raised above normal body temperature, such that the mean temperature of the tumor cells does not exceed a temperature of about 55° C., more preferably from about 40° C. to about 50° C., even more preferably from about 40° C. to about 44° C., and most preferably about 42.5. 1° C.

In other preferred embodiments, the tumor cells are cooled below normal body temperature such that the mean temperature of the tumor cells is within the range of from about −5° C. to about 36.5° C., more preferably from about 10° C. to about 30° C., and even more preferably from about 15° C. to about 25° C.

The oxidative environment stressor can be the application to the tumor cells of solid, liquid or gaseous oxidizing agents. Preferably, it involves exposing the tumor cells to a mixture of medical grade oxygen and ozone gas, most preferably by bubbling through the tumor cells, at the aforementioned temperature range, a stream of medical grade oxygen gas having ozone as a minor component therein. The ozone content of the gas stream and the flow rate of the gas stream are preferably selected such that the amount of ozone introduced to the tumor cells, either on its own or in combination with other stressors, does not give rise to excessive levels of cell damage such that the therapy is rendered ineffective.

Suitably, the gas stream has an ozone content of up to about 300 μg/ml, preferably up to about 100 μg/ml, more preferably about 30 μg/ml, even more preferably up to about 20 μg/ml particularly preferably from about 10 μg/ml to about 20 μg/ml, and most preferably about 14.5. 1.0 μg/ml. The gas stream is suitably supplied to the tumor cells at a rate of up to about 2.0 litres/min, preferably up to about 0.5 litres/min, more preferably up to about 0.4 litres/min, even more preferably up to about 0.33 litres/min, and most preferably about 0.24. 0.024 litres/min, at STP. The lower limit of the flow rate of the gas stream is preferably not lower than 0.01 litres/min, more preferably not lower than 0.1 litres/min, and even more preferably not lower than 0.2 litres/min.

The electromagnetic emission stressor is suitably applied by irradiating the tumor cells under treatment from a source of an electromagnetic emission while the tumor cells are maintained at the aforementioned temperature and while the oxygen/ozone gaseous mixture is being bubbled through the tumor cells. Preferred electromagnetic emissions are selected from photonic radiation, more preferably UV, visible and infrared light, and even more preferably UV light. The most preferred UV sources are UV lamps emitting primarily UV-C band wavelengths, i.e., at wavelengths shorter than about 280 nm. Such lamps may also emit amounts of visible and infrared light.

Ultraviolet light corresponding to standard UV-A (wavelengths from about 315 to about 400 nm) and UV-B (wavelengths from about 280 to about 315) sources can also be used. For example, an appropriate dosage of such UV light, applied simultaneously with the aforementioned temperature and oxidative environment stressors, can be obtained from lamps with a combined power output of from about 10 to about 30 watts, arranged to surround the sample container holding the tumor cells, each lamp providing an intensity, at a distance of 16 millimeters, of from about 5 to 20 mW/cm². Up to 8 such lamps, surrounding the sample container, with a combined output at 253.7 nm of 10 to 30 watts operated at an intensity to deliver a total UV light energy at the surface of the tumor cells of from about 0.025 to about 10 joules/cm², preferably from about 0.1 to about 3.0 joules/cm², may advantageously be used. Preferably, four such lamps are used.

The time for which the tumor cells are subjected to the stressors is normally within the time range of up to about 60 minutes. The time depends to some extent upon the chosen intensity of the electromagnetic emission, the temperature, the concentration of the oxidizing agent and the rate at which it is supplied to the tumor cells. Some experimentation to establish optimum times may be necessary on the part of the operator, once the other stressor levels have been set. Under most stressor conditions, preferred times will be in the approximate range of from about 2 to about 5 minutes, more preferably about 3 or about 3. minutes. The starting tumor cell temperature, and the rate at which it can be warmed or cooled to a predetermined temperature, tends to vary from subject to subject. Such a treatment provides modified tumor cells which are ready for injection into the subject.

In the practice of the preferred process of the present invention, the tumor cells may be treated with the stressors using an apparatus of the type described in U.S. Pat. No. 4,968,483 to Mueller. The tumor cells are placed in a suitable, sterile, UV light-transmissive container, which is fitted into the machine. The UV lamps re switched on for a fixed period before the gas flow is applied to the tumor cells providing the oxidative stress, to allow the output of the UV lamps to stabilize. The UV lamps are typically on while the temperature of the tumor cells is adjusted to the predetermined value, e.g., 42.5. 1° C. Then the oxygen/ozone gas mixture, of known composition and controlled flow rate, is applied to the tumor cells, for the predetermined duration of up to about 60 minutes, preferably 2 to 5 minutes and most preferably about 3 minutes as discussed above, so that the tumor cells experience all three stressors simultaneously. In this way, tumor cells are appropriately modified according to the present invention to achieve the desired effects.

A subject preferably undergoes a course of treatments, such individual treatment comprising removal of tumor cells, treatment thereof as described above, preferably after culturing the cells to increase the numbers thereof to a suitable value for use in the stressing apparatus, and re-administration of the treated tumor cells to the subject. A course of such treatments may comprise daily administration of treated tumor cells for a number of consecutive days, or may comprise a first course of daily treatments for a designated period of time, followed by an interval and then one or more additional courses of daily treatments.

In one preferred embodiment, the subject is given an initial course of treatments comprising the administration of 4 to 6 aliquots of treated tumor cells. In another preferred embodiment, the subject is given an initial course of therapy comprising administration of from 2 to 4 aliquots of treated tumor cells, with the administration of any pair of consecutive aliquots being either on consecutive days, or being separated by a rest period of from 1 to 21 days on which no aliquots are administered to the patient, the rest period separating one selected pair of consecutive aliquots being from about 3 to 15 days. In a more specific, preferred embodiment, the dosage regimen of the initial course of treatments comprises a total of three aliquots, with the first and second aliquots being administered on consecutive days and a rest period of 11 days being provided between the administration of the second and third aliquots. In the method of the invention, it is preferred that no more than one aliquot is administered to the subject on any given day.

It may be preferred to subsequently administer additional courses of treatments following the initial course of treatments. Preferably, subsequent courses of treatments are administered at least about three weeks after the end of the initial course of treatments. In one particularly preferred embodiment, the subject receives a second course of treatment comprising the administration of one aliquot of treated tumor cells every 30 days following the end of the initial course of treatments, for a period of 6 months.

It will be appreciated that the spacing between successive courses of treatments should be such that the positive effects of the treatment of the invention are maintained, and may be determined on the basis of the observed response of individual subjects. It is appreciated that tumor cells may be treated and preserved for later use as “booster” treatments.

Modes of Administration and Dose

The compositions of this invention may be administered to the subject at any site, particularly a site that is “distal” to or “distant” from the primary tumor.

The route of administration of a pharmaceutical composition may be parenteral, intramuscular, subcutaneous, intradermal, intraperitoneal, intranasal, via an afferent lymph vessel, or by another route that is suitable in view of the tumor being treated and the subject's condition. Intramuscular administration is preferred.

The dose given is an amount “effective” in bringing about a desired therapeutic response, be it the stimulation of an immune response, or the treatment of cancer as defined elsewhere in this disclosure. For the pharmaceutical compositions of this invention, effective doses typically fall within the range of about 10⁵ to 10¹¹ cells, including tumor cells and other cells from the subject being treated, if present. Preferably, between about 10⁶ to 10¹⁰ cells are used; more preferably between about 1×10⁷ and 2×10⁹ cells are used; more preferably between about 5×10⁷ and 2×10⁹ cells are used; even more preferably between about 1×10⁸ and 1×10⁹ cells are used. Multiple doses when used in combination to achieve a desired effect each fall within the definition of an effective amount.

The various components of the cellular vaccine are present in an “effective combination,” which means that there are sufficient amounts of each of the components for the vaccine to be effective. Preferably, at least about 10⁶, more preferably at least about 10⁷ but no more than 10¹⁰ tumor cells are present. Preferably, at least about 10⁵, more preferably at least about 10⁶, and still more preferably about 10⁷ but generally less than 10⁸ and typically less than 5×10⁷ tumor cells, tumor cell progeny, or the equivalents thereof are present. Any number of component cells or other constituents may be used, as long as the vaccine is effective as a whole. This will also depend on the method used to prepare the vaccine, such as whether the tumor cells are cultured before administration.

The pharmaceutical compositions of this invention may be given following, preceding, in lieu of, or in combination with, other therapies relating to generating an immune response or treating cancer in the subject. For example, the subject may previously or concurrently be treated by chemotherapy, radiation therapy, and other forms of immunotherapy and adoptive transfer. Where such modalities are used, they are preferably employed in a way or at a time that does not interfere with the immunogenicity of the compositions of this invention. The subject may also have been administered another vaccine or other composition in order to stimulate an immune response. Such alternative compositions may include tumor antigen vaccines, nucleic acid vaccines encoding tumor antigens, anti-idiotype vaccines, and other types of cellular vaccines, including cytokine-expressing tumor cell lines.

Timing of administration of compositions of this invention is within the judgment of the managing physician, and depends on the clinical condition of the patient, the objectives of treatment, and concurrent therapies also being administered. Typically, at an appropriate time in patient management, a first dose is given, and the patient is monitored for either an immunological or clinical response, often both. Suitable means of immunological monitoring include a one-way mixed lymphocyte reaction (“MLR”) using the patient's peripheral blood lymphocytes (“PBL”) as responders and primary tumor cells as stimulators.

An immunological reaction may also be manifest by a delayed inflammatory response at the injection site. Suitable means of monitoring of the tumor are selected depending on the tumor type and characteristics, and may include CT scan, magnetic resonance imaging (MRI), radioscintigraphy with a suitable imaging agent, monitoring of circulating tumor marker antigens, and the subject's clinical response. Additional doses may be given, such as on a monthly or weekly basis, until the desired effect is achieved. Thereafter, and particularly when the immunological or clinical benefit appears to subside, additional booster or maintenance doses may be given as required.

When multiple doses of a cellular vaccine are given to the same patient, some attention should be paid to the possibility that if allogeneic tumor cells are present in the vaccine, they may generate an anti-allotype response. The use of a mixture of allogeneic cells from a plurality of donors, and the use of different allogeneic cell populations in each dose, are both strategies that can help minimize the occurrence of an anti-allotype response. During the course of therapy, the subject is evaluated on a regular basis for side effects at the injection site, or general side effects such as a febrile response. Side effects are managed with appropriate supportive clinical care.

The compositions of this invention may optionally include a pharmaceutically acceptable excipient. Some examples of suitable excipients include sterile water, sterile saline, phosphate buffered saline, and the like.

Kit of Parts

The compositions of the present invention, and subcomponents thereof may be supplied in unit dosage or kit form. Kits of this invention may comprise various components of a cellular vaccine or pharmaceutical composition that are provided in separate containers. The containers may separately contain untreated tumor cells, tumor cells treated according to the method of this invention or adjuvant or pharmaceutically acceptable excipient, such that when mixed together they constitute a vaccine of this invention in unit dosage or multiple dosage form. They may also contain suitable devices, such as a syringe and a needle for delivering the composition to a patient.

Preferred kits comprise in separate containers: a tumor cell mixture treated according to the method of this invention in one container and a pharmaceutical excipient in another container. Packaged compositions and kits of this invention typically include instructions for storage, preparation and administration of the composition.

### EXAMPLES

The ability of stressed tumor cells prepared according to the preferred embodiments of the invention, to exhibit an immunogenic response to other tumor cells in the patient, and hence exhibit a cytotoxic effect thereon, can be assessed in vitro by assaying various markers and other characteristics of the stressed cells on culturing them.

For example, it has been reported (Ronchetti, Anna et. al., “The Journal of Immunology,” 1999, 163:130-136) that the immune response elicited by tumor cells inversely correlates to the presence of professional phagocytes and that cells undergoing apoptosis may elicit an immune response and be immunogenic in vivo. Also, it has been reported (Henry, Frederic et. al., “Cancer Research” 59, 3329-3332, Jul. 15, 1999) that tumor-bearing rats are cured with an 80% success rate by injection of antigen-presenting cells that had phagocytosed apoptotic bodies derived from poorly immunogenic tumor cells, whereas phagocytic cells exposed to non-apoptotic tumor cell extracts are essentially without effect.

Consequently, experiments were performed to demonstrate apoptosis in the stressed melanoma cells of the invention (Example 1), and in breast cancer cells (Example 2) by the known Annexin V staining technique.

In addition, the cell surface molecule CD95 is a pro-apoptotic marker, upregulation of which indicates an increased tendency of such cells towards apoptosis (Sibiryak, S V et. al., “Russ. J. Immunol.” 1. 1999; Lorenzo, E. et. al., PubMed Abstract 1179855). Thus the likelihood of an immunogenic response of such stressed tumor cells toward autologous tumor cells in a mammalian patient's body can be assessed by investigating CD-95 expression in stressed cells prepared according to the invention Accordingly, also as reported in Example 1 below, experiments were conducted to quantify the expression of CD95 in such stressed HTB-76 melanoma cells. As reported in Example 2, similar experiments were conducted with breast cancer cells.

Cell surface molecule CD54 (also known as ICAM-1) is an “adhesion molecule” which plays a role in the interaction of NK cells with a variety of tumor cells including carcinoma and melanoma (Eisenthal, Avi et. al., “Pathobiology” 1998; 66: 205-208). Increased expression of CD-54 on stressed tumor cells of the present invention is thus another indicator of immunogenicity of these autologous stressed tumor cells towards the host tumor in vivo, and this was determined as described in Example 3 below.

Enhanced expression of the cell surface molecules HLA-DR (also known as MHC-class II) and CD-54 by melanoma cells has been reported to be associated with zones of T-cell infiltration whereas no such expression (or diminished expression) is observed in relatively unaffected regions of tumors (Murphy, George et. al., “The Journal of Investigative Dermatology”, 100:335S-341S, 1993). Moreover, expression of HLA-DR is reportedly dramatically increased in a tumor vaccine using TNFα gene transduction (Li, Biaoru et. al., “In Vivo” 13, 433-438 (1999)). Upregulation of HLA-DR and CD-54 on the stressed cells of the present invention accordingly is a further indicator of immunogenicity of these autologous stressed cells towards the host tumor. This is also explored in Example 3 below.

Apoptotic tumor cells as produced by the process of this invention, evidenced as described in the Examples below, have upregulated MHC-II ex vivo, so as to become an immunogenic complex. This is also explored in Example 3 below. While it is not intended that the invention should be limited to any particular theory or mode of action, it is believed that, on re-introduction into the tumor-bearing mammalian patient, cells with upregulated MHC-II will activate dendritic cells by binding to them in vivo, so that the dendritic cells undergo maturation, in the lymph nodes interact with T-helper cells, to produce specific Tc (T-cytotoxic) cells which will attack the tumor cells in the patient's body. This is believed to be another mechanism whereby the stressed autologous cells produced in the present invention may act as an autologous cancer vaccine.

Methods

HTB-67 Melanoma cells and MCF-7 breast cancer adherent cells were used in the experiments described below in Examples 1-4. For cell culture, the cells were grown in a 37 degrees C. incubator with humidified air containing 5% carbon dioxide and sub-cultured with Minimum Essential Medium (item 41500, Gibco) supplemented with 10% fetal calf serum, 1 mM sodium pyruvate and 1.5 g/L sodium bicarbonate. MCF-7 adherent cells were harvested with trypsin at 95% confluence during exponential growth phase and counted using a haemocytometer. HTB-67 suspension cells were harvested at 5×10⁵ cells/ml.

### Example 1

The HTB-67 melanoma cell line was harvested and 2×10⁶ cells were placed in 12 mls of culture medium with 3% BSA. These cells were then subjected to oxidative stress, namely bubbling of ozone/oxygen (14.5±1.0 μg/ml ozone) mixture through the suspension at a rate of about 0.24 litres per minute, with simultaneous exposure to UV light having a major wavelength component of 253.7 nm, for 3 minutes, at about 42.5 degrees C. for 3 minutes, using an apparatus generally as described in U.S. Pat. No. 4,968,483 Mueller. After the stress treatment, 1.8×10⁶ cells were obtained from the container. Other, control cells were also harvested but were not stressed.

5×10⁵ control cells and a similar number of stressed cells were separately incubated with 1.6 μl of anti-CD-95 (APO/Fas) FITC (Idlabs) for 45 minutes on ice in 80 μl of pbs. During this time, the cells were stained with 1.25 μl of Annexin-V in 500 μl binding buffer, then stained with 10 μl of propidium iodide o-Alert, BD Sciences). In addition, one stressed sample stained with Annexin V only and one with propidium iodide only were prepared, in order to properly set compensation values on the flow cytometer during acquisition. All samples were washed and strained before acquisition.

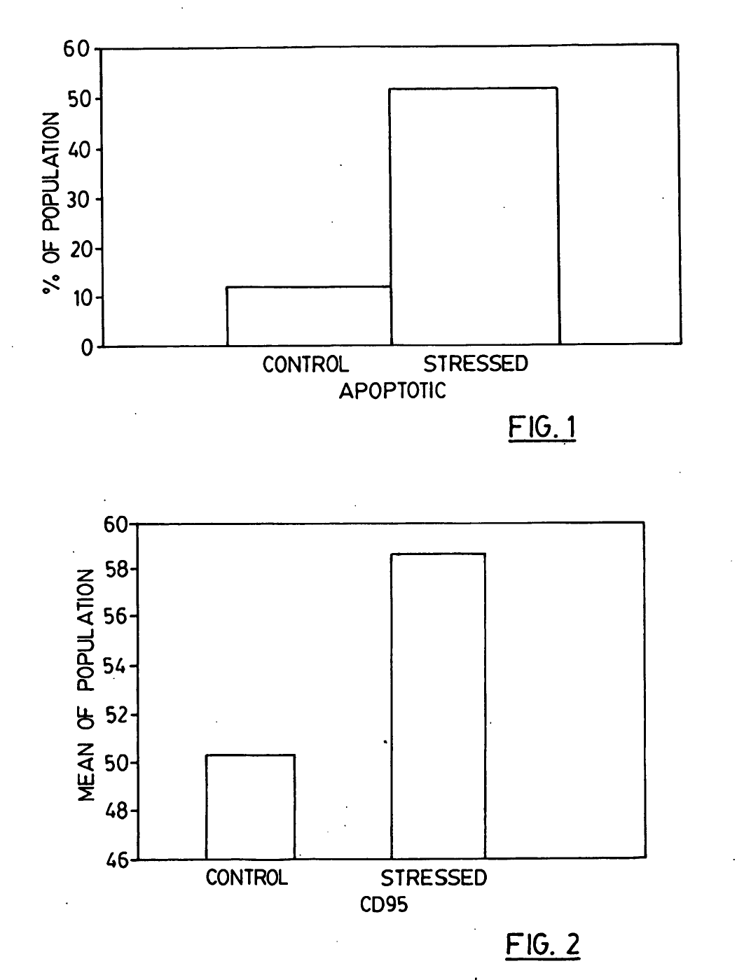

The stressed cells showed an apoptotic fraction that was 4.3 fold larger than the control sample. The population of apoptotic stressed cells was 51.34% versus 11.94% in the control population. These results are shown graphically on accompanying FIG. 1. These values were determined by separating a plot of Annexin-V versus PI fluorescence into four quadrants, thereby showing the distribution of normal, apoptotic, necrotic and late necroticlapoptotic cells. These quadrants were set using the control cells as a guide. Anti-CD-95 (APO/Fas) staining was analyzed by calculating the mean fluorescent intensity (fluorescent counts) for 99% of the gated population of cells. The cells showed an upregulation of CD95 from a mean of 50.54 in the control sample to 54.38 in the stressed sample. Thus results are presented graphically on FIG. 2.

### Example 2

The MCF-7 breast cancer cell line was harvested and 2×10⁶ cells were placed in 12 mls of culture media with 3% BSA. These cells were stressed as described in Example 1. After stressing, 5×10⁵ of each of stressed cells and control cells were incubated with 1.6 μl of anti-CD-95 (APO/Fas) for 45 minutes on ice in 80 ∥l of PBS. The staining with Annexin V and measurements proceeded as in Example 1.

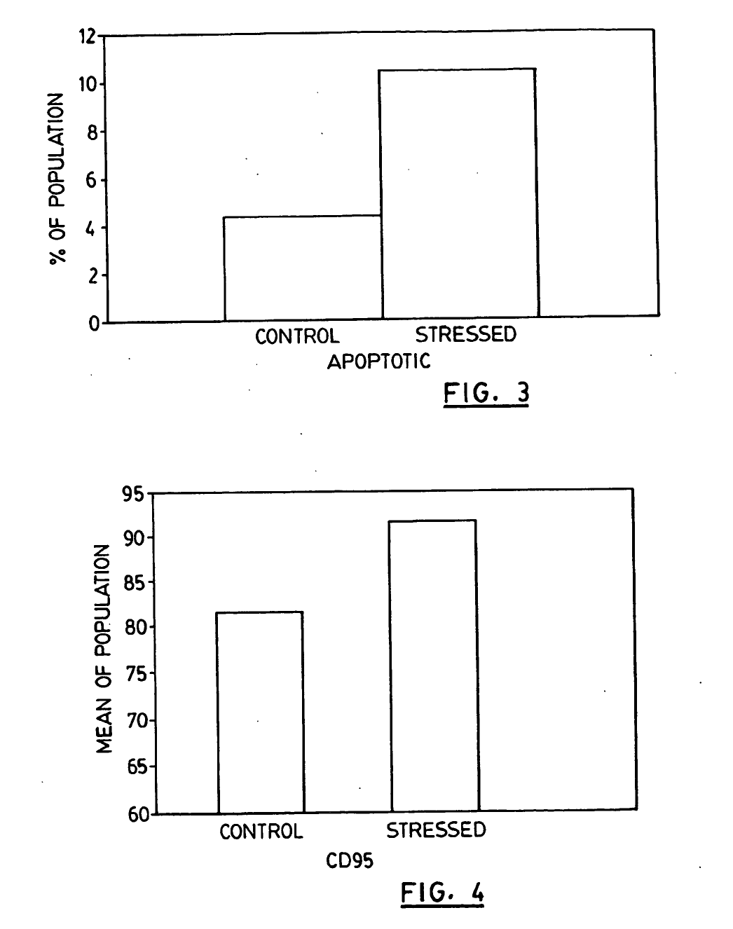

The stressed cells showed an apoptotic fraction that was 2.4 fold larger than the control sample. The population of apoptotic cells in the treated sample was 10.39% compared with 4.35% in the control sample. These Annexin V staining results are shown graphically on FIG. 3. The results for CD95 expression, measured as described in Example 1 are shown graphically on FIG. 4. The gated MCF-7 cells showed an upregulation of CD95 from a mean of 69.95 in the control sample to 74.09 in the stressed sample.

### Example 3

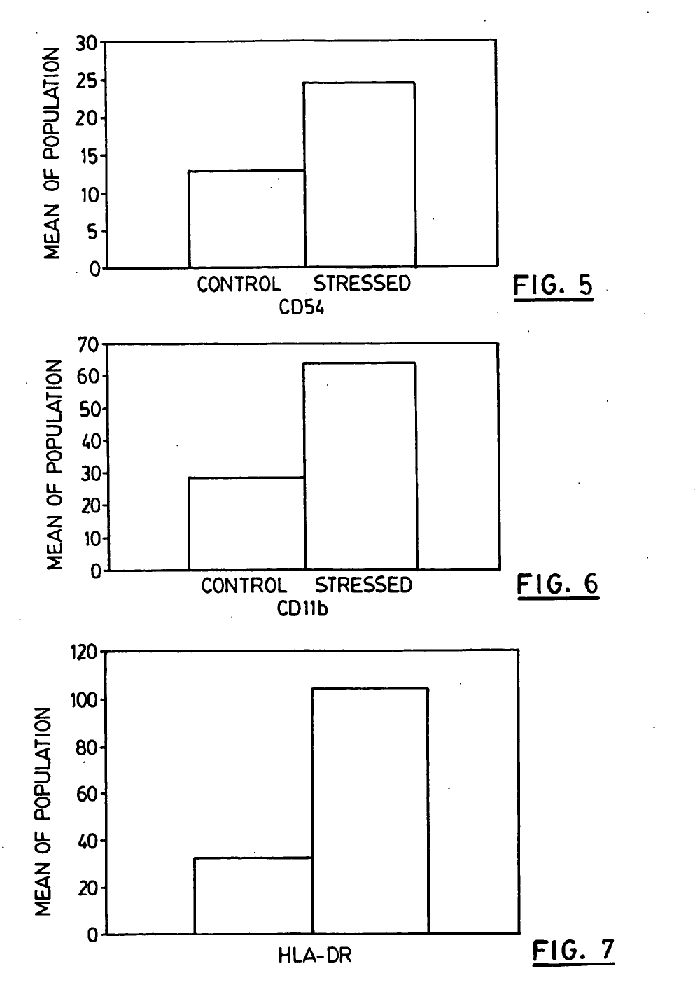

In a similar manner to that described above, stressed and control HTB-6 melanoma cells prepared as described above were treated with antibodies to CD-11b, CD-54 and HLA-DR, stained and analyzed for upregulated expression of each of these surface molecules. FIG. 5 of the accompanying drawings presents the CD-54 results graphically, and shows that the treated cells have a mean population of cells expressing CD-54 of almost 25%, compared with less than 13% in the control population.

FIG. 6 of the accompanying drawings similarly presents graphically the CD-11b results. In this case the population of stressed cells expressing CD-11b is about 64%, compared with about 29% of the control cells.

FIG. 7 of the accompanying drawings similarly presents graphically the HLA-DR results. In this case the population of stressed cells expressing HLA-DR is 100%, compared with about 32% of the control cells.

### Example 4

The effects of the tumor vaccine prepared according to the methods of this invention may be further studied by the growth of the highly aggressive melanoma tumor arising from mouse melanoma B16-F10 grafted subcutaneously in C57B16/J mice.

B16-F10 melanoma tumor cells are maintained in sterile culture by standard techniques. The vaccine is prepared from a suspension of these cells in saline or other suitable medium, at a concentration of 1×10⁷ to 5×10⁷ per ml. Ten ml of the cell suspension is stressed according to the methods of this invention described above.

Mice are randomized into the following groups: 1) untreated; 2) cell suspension medium treated; 3) vaccine treated. Animals in groups 2 and 3 are treated on days 1, 2 and 14 or 1, 14 and 28 with either 0.05 ml of cell suspension medium (group 2) or 0.05 ml of vaccine containing about 2×10⁷ processed cells suspended in cell suspension medium. One day following the last injection, mice are injected with a single subcutaneous injection into the flank containing 5×10⁵ viable B16-F10 tumor cells per animal.

Animals are examined three times per week for signs of a tumor. Tumor growth is measured using a caliper, and mice are sacrificed when the tumors reach a diameter of 15 mm. Histological studies are performed.

### Example 5

Colon carcinoma cells for use according to the present invention can be obtained, but only in very small amounts, by biopsy of a human patient having such a tumor. The malignant cells so obtained can be cultured in vitro to obtain a suitable amount for stressing as described, and then recovered, suspended and administered to the patient from whom the initial cell samples were obtained. An alternative is in vivo growth and multiplication of the originally obtained tumor cells from the patient. For this, a suitable animal model such as SCID mouse may be used. The cells are injected into SCID mice, allowed to multiply therein and then recovered from the mouse when a suitably large quantity has been obtained in this way. It may be necessary to use more than one generation of SCID mice, in order to grow the appropriate numbers of colon carcinoma cells. Harvesting, purification, treatment and injection of the stressed cells to the original patient is conducted as described above. These techniques of growing human tumor cells in appropriate animal models are known in the art.

The examples are presented as a further guide to a practitioner of ordinary skill in the art, and are not meant to be limiting in any way.

1. A pharmaceutical composition for administration to a mammal suffering from a malignancy, comprising mammalian tumor cells selected from melanoma cells, colon carcinoma cells and breast-cancer tumor cells, the mammalian tumor cells having been treated ex vivo by stressing with an oxidative environment and UV light simultaneously, so as to render the tumor cells effective to elicit an immune response to a melanoma, colon carcinoma or breast cancer tumor respectively, in said mammal.

2. The pharmaceutical composition of claim 1 wherein the tumor cells are autologous cells.

3. The pharmaceutical composition of claim 1 wherein the oxidative environment comprises applying an oxidizing agent to the tumor cells.

4. The pharmaceutical composition of claim 3 wherein the oxidizing agent contains ozone gas.

5. The pharmaceutical composition of claim 4 wherein the oxidizing agent comprises a mixture of ozone gas and medical grade oxygen, the ozone gas being contained in the mixture in a concentration of up to about 300 μg/ml.

6. The pharmaceutical composition of claim 5 wherein the ozone gas is contained in the mixture in a concentration of up to about 30 g/ml.

7. The pharmaceutical composition of claim 1 wherein the tumor cells are additionally subjected extracorporeally to a temperature stressor.

8. The pharmaceutical composition of claim 7 wherein the mean temperature at which the tumor cells are stressed is in the range of from about 37° C. to about 44° C.

9. The pharmaceutical composition of claim 1 wherein the tumor cells are stressed in suspension in a volume of up to about 400 ml.

10. The pharmaceutical composition of claim 9 wherein the tumor cells are subjected to the stressors for a period of up to about 60 minutes.

11. The pharmaceutical composition of claim 7 wherein the tumor cells are simultaneously subjected to said oxidative environment, said UV light and said temperature stressor.

12. The pharmaceutical composition of claim 1 wherein the mammal is a human.

13. A kit of parts comprising:
(a) colon carcinoma, melanoma or breast cancer tumor cells treated ex vivo with at least two stressors selected from the group consisting of an oxidative environment, thermal stress and UV light; and
(b) a pharmaceutically acceptable excipient.

14. The kit of parts of claim 13 further comprising a syringe and needle.

15. (canceled)

16. (canceled)

17. A method for treating a mammal suffering from melanoma, comprising administering to said mammal a composition of mammalian melanoma cells treated ex vivo by stressing said melanoma cells simultaneously with an oxidative environment and UV light, wherein said composition of treated melanoma cells is administered in an amount effective to elicit an immune response in said mammal.

18. The method of claim 17, wherein said melanoma cells are additionally subjected excorporeally to a temperature stressor.

19. The method of claim 17, wherein said melanoma cells are autologous.

20. A method for treating a mammal suffering from colon carcinoma, comprising administering to said mammal a composition of mammalian colon carcinoma cells treated ex vivo by stressing said colon carcinoma cells simultaneously with an oxidative environment and UV light, wherein said composition of treated colon carcinoma cells is administered in an amount effective to elicit an immune response in said mammal.

21. The method of claim 20, wherein said colon carcinoma cells are additionally subjected excorporeally to a temperature stressor.

22. The method of claim 20, wherein said colon carcinoma cells are autologous.

23. A method for treating a mammal suffering from breast cancer, comprising administering to said mammal a composition of mammalian breast cancer cells treated ex vivo by stressing said breast cancer cells simultaneously with an oxidative environment and UV light, wherein said composition of treated breast cancer cells is administered in an amount effective to elicit an immune response in said mammal.

24. The method of claim 23, wherein said breast cancer cells are additionally subjected excorporeally to a temperature stressor.

25. The method of claim 23, wherein said breast cancer cells are autologous.

26. An immunogenic tumor cell composition, comprising tumor cells treated ex vivo to produce modified tumor cells effective to elicit, in a mammalian subject, an immune response to an unmodified mammalian tumor cell derived from a cancer selected from the group consisting of colon carcinoma, melanoma and breast cancer.

27. The composition of claim 26, wherein said tumor cells are treated simultaneously with an oxidative environment and UV light.

28. The immunogenic tumor cell composition of claim 26, wherein said treated tumor cells are human colon carcinoma cells and said cancer is colon carcinoma.

29. The immunogenic tumor cell composition of claim 26, wherein said treated tumor cells are human melanoma cells and said cancer is melanoma.

30. The immunogenic tumor cell composition of claim 26, wherein said treated tumor cells are human breast cancer cells and said cancer is breast cancer.
